(19) United States
(12) Patent Application Publication
(10) Pub. No.: US 2012/0004323 A1
(43) Pub. Date: Jan. 5, 2012

(54) IMPLANT PROCESSING METHODS FOR THERMALLY LABILE AND OTHER BIOACTIVE AGENTS AND IMPLANTS PREPARED FROM SAME

(76) Inventors: Peter Markland, Birmingham, AL (US); Thomas R. Tice, Indian Springs, AL (US)

(21) Appl. No.: 13/173,021

(22) Filed: Jun. 30, 2011

Related U.S. Application Data
(60) Provisional application No. 61/360,150, filed on Jun. 30, 2010.

(51) Int. Cl. A61K 47/30; B29C 47/00; A61K 9/32

(52) U.S. Cl. 514/772.3; 427/2.14; 264/176.1

(57) ABSTRACT

Disclosed herein are processes for preparing implants that are particularly useful for thermally labile bioactive agents but can also generally be used with any bioactive agent. The disclosed processes avoid the use of heat during processing and therefore avoid heat induced degradation of the bioactive agent. Also disclosed are implants prepared by the disclosed methods.

### CROSS-REFERENCE TO RELATED APPLICATIONS

This application claims the benefit of priority from prior U.S. Provisional Application No. 61/360,150, filed Jun. 30, 2010, the entire contents of which are incorporated into this application by reference.

### BACKGROUND

Implants often comprise a biodegradable or bioresorbable polymeric matrix with a bioactive agent or drug dispersed throughout or localized within this polymeric matrix. These implants can be of various sizes and shapes such as cylinders or spheres. One function of an implant can be to release bioactive agents or drugs from its polymeric matrix in a controlled manner. There are a number of different mechanisms that bioactive agents and drugs can release from implants providing a number of different release profiles. Once administered to a subject, an implant can provide a prolonged release or extended release of a bioactive agent or drug for days or even months for the treatment of a variety of therapeutic indications. The implants can be used for systemic treatment or local treatment.

Implant materials and methods of making implants are compatible with most classes of drugs and bioactive agents. For example, one common method to make implants is by heat extrusion. Some bioactive agents and drugs, however, are difficult to formulate into an implant by heat extrusion because the heat can adversely degrade them or alter their physical and biological properties. Accordingly, a need exists for improved implant processing methods and compositions that are compatible with temperature sensitive bioactive agents and drugs. These needs and other needs are satisfied by the present invention.

### SUMMARY

Disclosed herein are processes for preparing implants that are particularly useful for thermally labile bioactive agents but can also generally be used with any bioactive agent. The disclosed processes avoid the use of heat during processing and therefore avoid heat induced degradation or other alteration of the physical or biological activity of the bioactive agent. Particularly, the disclosed processes are carried out at or below 70° C. to effectively avoid heat degradation or alteration of other properties of the bioactive agent.

Also disclosed herein are bioresorbable implants prepared by the processes of the invention.

Also disclosed are bioresorbable implants comprising (a) a bioresorbable polymer matrix; (b) a bioactive agent dispersed in the matrix; and (c) from about 0.05% to about 5% of a plasticizing agent in the matrix.

### DETAILED DESCRIPTION

In this specification and in the claims that follow, reference will be made to a number of terms that shall be defined to have the following meanings:

The word “comprise,” or variations such as “comprises” or “comprising,” will be understood to imply the inclusion of a stated component, integer, step, or group of components, integers, or steps but not the exclusion of any other component, integer, or step or group of components, integers, or steps, unless stated otherwise.

The singular forms “a,” “an” and “the” include plural referents unless the context clearly dictates otherwise. Thus, for example, reference to “a bioactive agent” includes mixtures of two or more such agents.

A weight percent of a component, unless specifically stated to the contrary, is based on the total weight of the formulation or composition in which the component is included.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “implant,” as used herein, refers to any article that is greater than 1 mm in length in at least one dimension of the implant. In a further aspect, the device has one dimension that is from 1 mm to 50 mm, 1.2 mm to 45 mm, 1.4 mm to 42 mm, 1.6 mm to 40 mm, 1.8 mm to 38 mm, or 2.0 mm to 36 mm, 5.0 mm to 33 mm, or 10 mm to 30 mm. In a further aspect, the device has one dimension that is greater than 3 cm, even up to or greater than 10 cm, 20 cm, or even 30 cm. The implant can have any suitable diameter, for example, from 1 mm to 50 mm.

The term “bioresorbable,” as used herein, refers to a substance that can be safely excreted from a subject, such as a human. A “bioresorbable” substance can, but does not necessarily, biodegrade or bioerode.

The term “biodegradable,” as used herein, refers to a material that will erode to soluble species or that will degrade under physiologic conditions to smaller units or chemical species that are, themselves, non-toxic (biocompatible) to the subject and capable of being metabolized or eliminated from the subject.

The term “microparticle,” as used herein is used herein to include nanoparticles, microspheres, nanospheres, microcapsules, nanocapsules, and particles, in general. As such, the term microparticle refers to particles having a variety of internal structure and organizations including homogeneous matrices such as microspheres (and nanospheres) or heterogeneous core-shell matrices (such as microcapsules and nanocapsules), porous particles, multi-layer particles, among others. The term “microparticle” refers generally to particles that have sizes in the range of about 10 nanometers (nm) to about 2 mm (millimeters) or larger.

A “bioactive agent,” as used herein refers to an agent that has biological activity. The bioactive agent can be used to treat, diagnose, cure, mitigate, prevent (i.e., prophylactically), ameliorate, modulate, or have an otherwise favorable effect on a disease, disorder, infection, and the like. Bioactive agents also include those substances which affect the structure or function of a subject, or a pro-drug, which becomes bioactive or more bioactive after it has been placed in a predetermined physiological environment.

“Extrusion,” as used herein, refers to any extrusion process and generally includes tabletting processes, which are referred to herein as “press extrusion.”

To avoid heat degradation or altering other properties like physical and biological properties of bioactive agents, the disclosed processes utilize methods that do not involve the use of heat. Specifically, the disclosed processes are preferably carried out at a temperature no greater than about 70° C. In some aspects, the processes are preferably carried out at 65° C., 60° C., 55° C., 50° C., 45° C., 40° C., 30° C., or at room temperature or below. In further aspects, the processes are carried out at or below 27° C., or at or below 26° C. or 25° C. The lower end of the temperature range used during processing can vary widely. For example, in some aspects, the processes are carried out at a temperature range of from 0° C. to 27° C., from 10° C. to 27° C., from 15° C. to 27° C., or from 20° C. to 27° C. Such temperature ranges will generally avoid heat degradation or other alteration of thermally labile bioactive agents and other bioactive agents.

In the processes of the invention, a bioresorbable polymer and a solid bioactive agent are mixed to form an admixture. This can include dry-admixing the bioresorbable polymer and solid bioactive agent. This can also include forming a solution of the bioactive agent and spraying or coating this solution onto the ground bioresorbable polymer followed by optionally drying the sprayed or coated polymer or microparticle.

The biodegradable polymer or microparticle can be mixed with the bioactive agent using a variety of methods. Commercially available mixers can be used, particular in connection with a large-scale process. In smaller scale processes, the bioresorbable polymer can be mixed with the solid powdered bioactive agent using a mortar and pestle.

The admixing step is carried out without the use of heat, as discussed above. During the admixing step, the bioresorbable polymer and the bioactive agent can be dry, that is, a dry admixing step is carried out. If dry admixing is used, neither the polymer or the bioactive agent contains any appreciable amount of solvent, e.g., 0.1% or less, including 0%.

Alternatively, the polymer can be plasticized with a plasticizing agent prior to the admixing step and thus may contain residual plasticizing agent, which can include various solvents, liquids, gases, and polymers. Additionally, a plasticizing agent can be added to the polymer and/or bioactive agent during the admixing step itself. In another aspect, when the bioresorbable polymer is in the form of a microparticle, this microparticle may contain solvent within the microparticle matrix and may release this solvent as a plasticizing agent during the admixing step or during another step of the process. The residual solvent in the microparticle may function as a plasticizing agent during the processing of the implant.

When the bioactive agent is sprayed or coated onto the polymer or microparticle as a solution, the resulting sprayed or coated polymer or microparticle can optionally be dried. The drying step, if used, however, should be carried out without the use of heat. For example, the sprayed or coated polymer or microparticle can be dried at room temperature or below. The solvent that is used for the solution of the bioactive agent can also function as a plasticizing agent for the polymer, as discussed above.

A plasticizing agent can be used in the disclosed processes in order to plasticize the polymer and thereby improve the polymer/microparticle and bioactive agent mixing process as well as improving the adhesive strength of the implant. For example, the plasticizing agent can reduce the chance of the implant breaking apart or crumbling during routine handling. Additionally, a plasticizing agent can also prolong the release time of the bioactive agent from the device relative to an implant prepared without the use of a plasticizing agent. The plasticizing agent can also increase the release rate of the bioactive agent (i.e., decrease the release time (decrease the overall duration of release)).

The plasticizing agent can be added to the polymer or the admixture that includes the bioactive agent. Thus, the plasticizing agent can be added at any point in the process prior to or even during the processing step, such as the extruding or molding step. The plasticizing agent can be added to the polymer or admixture as a liquid, gas, or polymer, for example, by exposing the polymer or admixture to the liquid, vapor, or polymer used as the plasticizing agent.

The plasticizing agent can be a variety of low melt binders or a compression-based binders, including a variety of solvents. Preferably, the solvent is a solvent for the polymer or the polymer of the microparticle. That is, the polymer is at least partially soluble in the plasticizing agent. In some aspects, the plasticizing agent can be an organic solvent, such as methylene chloride, ethylene chloride, chloroform, ethyl acetate, acetone, ethanol, methanol, isopropanol, butyl alcohol, dimethyl sulfoxide, or a mixture thereof. Certain drugs that are used as the bioactive agent can also be the plasticizer.

In addition, various other polymers which are admixed or blended with the bioresorbable polymer can be used. Polymeric plasticizers would themselves have a lower T_{g} such that the resulting admixture would have a T_{g} that is lower than that of the un-blended bioresorbable polymer. Suitable polymers for use as plasticizing agents include various viscous bioresorbably polymers. Examples include those polymers disclosed in U.S. Patent Application publication no. 2009/0124535 entitled “VISCOUS TERPOLYMERS AS DRUG DELIVERY PLATFORM” to Markland et al, which is incorporated herein by reference for its teachings of terpolymers and methods of making them. Further examples of polymers suitable for use as plasticizing agents include viscous poly(hexyl-lactide), or polymers made from mono- or di-hexyl substituted glycolide or lactide.

Still further examples of suitable polymeric plasticizing agents generally include viscous biodegradable and biocompatible polyesters (including random copolymers of two or more hydroxyl acid monomers) having bulk viscosity of about 10,000 poise or less; and preferably about 4,000 poise or less. Still further examples include without limitation block copolymers containing one or more blocks of polyester monomers, and/or one or more blocks of a polyester and/or a hydrophilic block (such as PEG and/or PVP and/or polysaccharide).

Further examples of polymeric plasticizers include without limitation viscous biodegradable polymers that are linear polymers, branched polymers, star polymers, comb-shaped polymers, dendrimer polymers (and copolymers). Additives that permit extrusion at lower temperatures can include blends comprising one or more of such biodegradable polymer described above. Further, additives can include one or more biodegradable polymers described above with one or more additional additives such as plasticizers, solvents, lipids, oils, solutions, buffers, salts, soluble agents, and the like. The solid biodegradable polymers include homopolymers and copolymers comprising lactide, glycolide, caprolactone, hydroxybutyrates and generally any biocompatible and biodegradable hydroxy acids), including poly(lactide), PLG, and copolymers of lactide-caprolactone, lactide-glycolide-caprolactone, as well as copolymers comprising one or more of lactide, glycolide, and/or caprolactone, and one or more block of a hydrophilic polymer such as PEG or PVP.

In another aspect, neither a plasticizing agent nor a solvent is used at any point in the inventive process. According to this aspect, the implant should be prepared to a specified hardness to ensure that the implant will not break or crumble after manufacture or during administration. The implant can be characterized by hardness. A suitable hardness will vary depending on the composition of the implant but will generally be a hardness that prevents the implant from breaking apart or crushing during routine handling. For example, for certain implants, a hardness of at least 25 as measured by a PFIZER hardness tester can be suitable. Various other hardness values can also be exhibiting by the implants. The PFIZER hardness tester operates on the same mechanical principle as ordinary pliers. As the tablet is crushed in the jaws of the device, the force is recorded on a dial indicator. The dial indicator remains at the reading where the tablet breaks. It returns to zero when a reset button is pressed. The force is provided in units of pounds.

Once the bioactive agent and the polymer are thoroughly mixed, or when the bioactive agent has been otherwise applied to the polymer or microparticle (e.g., through spraying or coating), the admixture, coated, or sprayed polymer is processed, for example through extrusion, molding, or other processing, into an implant at a temperature that is preferably no greater than 70° C. This temperature in some aspects can be no greater than 55-60° C. In other aspects, the extrusion or molding step can be carried out at lower temperatures, such as room temperature or below, or 25-27° C. or below, as discussed above.

The processing of the implant can comprise a variety of procedures. For example, processing can generally include any type of extrusion or molding, including without limitation, melt pressing, injection molding, or press extruding using a tablet press. If melt pressing is used, the temperature is preferably kept at or below 55-60° C. In one aspect, the admixture or sprayed or coated polymer is processed using press extrusion with a tablet press. According this aspect, the admixture, sprayed, or coated polymer is added to a die which is sized according to the particular therapeutic application of the implant. Optionally, a plasticizing agent can be added to the die prior to pressing. Alternatively, this step can be carried out without the addition of a plasticizing agent. The press is applied to the die under pressure to form an implant in the shape of the dye. The implant can then be removed from the die.

In one aspect, the polymer used in the processes is either purchased in a sufficiently ground state or ground using a grinding mill, prior to forming the admixture or coating or spraying the bioactive agent onto the polymer or microparticle. When the polymer or microparticle is ground manually, the polymer or microparticle can be cooled at a temperature of −150° C. or less, e.g., using liquid nitrogen. The time to complete the cooling step will depend on the amount of the polymer to be cooled. Before placing the polymer in the grinding mill, the grinding mill can also be cooled at a temperature of −150° C. or less.

A variety of commercially available grinding mills can be used in this process. An example is a Retsch Mill ZM 100 (available from Retsch, Dusseldorf, Germany). When using the Retch Mill, the cooled or frozen polymer or microparticle can be continuously added to the mill and ground using an appropriate speed, for example, about 18,000 rpm.

After grinding the polymer, the polymer can optionally be filtered through appropriate size sieves in order to remove polymers of microparticles of a certain size. In one aspect, the polymer or microparticle can be sieved through both a sieve that ranges in size from 90 to 300 microns, including, for example, a 90-micron sieve and/or a 300-micron sieve.

The bioactive agent can in some aspects be in a powder or ground form, although other forms can be used, such as liquid bioactive agents or bioactive agent particles. Many such bioactive agents can be obtained commercially or can be processed using a grinding mill as discussed above. In addition, any other pharmaceutical processing technique can be used to process the bioactive agent accordingly, including techniques such as ball milling, jet milling, spray drying, and the like.

Once the implant is formed, a variety of post-manufacturing processes can be carried out. For example, the implant can be subjected to a fluid treatment that will effectively change the surface morphology of the implant and therefore alter the release profile. This process is described in detail in U.S. Patent Application publication No. 20060029637, “Methods for manufacturing delivery devices and devices thereof,” to Tice et al, which is incorporated herein by reference in its entirety for its teachings of fluid-treatment methods. A preferred aspect of the method involves dipping the implant in a solvent for the polymer for a short period of time (e.g., a few seconds). Preferably, the solvent does the solvent contain a polymer. Thus the implant is dipped in solvent-only. Preferred solvents for poly(lactide), poly(glycolide), poly(caprolactone), poly(lactide-glycolide), or a copolymer, combination, or mixture thereof include methylene chloride, ethylene chloride, chloroform, ethyl acetate, and mixtures thereof. In other aspects, the fluid-treatment step can include a solvent that contains an additional polymer (polymer solution), such that the additional polymer will be coated onto the surface of the implant.

Suitable bioresobable and/or biodegradable polymers for use with the invention include without limitation poly(lactide), a poly(glycolide), a poly(lactide-co-glycolide), a poly(caprolactone), a poly(orthoester), a poly(phosphazene), a poly(hydroxybutyrate) a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations thereof.

In some aspects, the bioresorbable or biodegradable polymer comprises one or more lactide residues. The polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When poly(lactide-co-glycolide), poly(lactide), or poly(glycolide) is used, the amount of lactide and glycolide in the polymer can vary. For example, the biodegradable polymer can contain 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

In a further aspect, the biodegradable polymer can comprise a poly(caprolactone) or a poly(lactide-co-caprolactone). For example, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

Any of the aforementioned polymers can be used to form the microparticle of the invention, if a microparticle is desired for use.

The bioactive agent can be present in the implant in any suitable amount. For example, the bioactive agent can be present in an amount ranging from 0.05% to 80% by weight of the implant, for example, 0.1%, 0.5%, 5%, 10%, 15%, 20%, 30%, 40%, 45%, 50%, 55%, 60%, 70%, or 80%.

Examples of bioactive agents that can be incorporated into the compositions of the invention include generally any bioactive agents and particularly, thermally-labile bioactive agents. Examples include without limitation small molecules, peptides, proteins such as hormones, enzymes, antibodies, receptor binding proteins, antibody fragments, antibody conjugates, nucleic acids such as aptamers, iRNA, siRNA, microRNA, DNA, RNA, antisense nucleic acid or the like, antisense nucleic acid analogs or the like, VEGF inhibitors, macrocyclic lactones, dopamine agonists, dopamine antagonists, low-molecular weight compounds, high-molecular-weight compounds, or conjugated bioactive agents.

Other bioactive agents can include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, antipsychotics, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Still other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents include, but are not limited to, peptide drugs, protein drugs, therapeutic antibodies, anticalins, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, anti-inflammatory agents, nonsteroidal anti-inflammatory agents, antiparkinsonian agents, antihypertensive agents, β-adrenergic blocking agents, nutritional agents, anti-TNF agents and the benzophenanthridine alkaloids. The agent can further be a substance capable of acting as a stimulant, sedative, hypnotic, analgesic, anticonvulsant, and the like.

Other bioactive agents include but are not limited to analgesics such as acetaminophen, acetylsalicylic acid, and the like; anesthetics such as lidocaine, xylocalne, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; antipsychotics such as clozapine, haloperidol, carbamazepine, gabapentin, topimarate, bupropion, sertraline, alprazolam, buspirone, risperidone, aripiprazole, olanzapine, quetiapine, ziprasidone, iloperidone, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxan®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, Ropinerole, Ivermectin, Moxidectin, Afamelanotide, Cilengitide, or a combination thereof. In one aspect, the bioactive agent can be a combination of Rifampicin (Rifampin in U.S.) and Minocycline.

Also disclosed herein are biodegradable implants comprising (a) a bioresorobable polymeric matrix or a biodegradable polymeric matrix; (b) a bioactive agent dispersed in the matrix; and (c) from about 0.05% to about 5% of a plasticizing agent in the matrix. The biodegradable implants can be prepared by the methods discussed above and can thus contain residual plasticizing agent from the process.

The implants of the invention can be administered to a subject to effectively deliver the bioactive agent to the subject. The subject can be a vertebrate, such as a mammal, a fish, a bird, a reptile, or an amphibian. The subject of the herein disclosed methods can be, for example, a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. Dosages and particular formulations can be determined by one of skill in the pharmaceutical arts and will vary widely depending on the indication being treated.

Various specific aspects of the invention are numbered 1-77 and are recited below.

1. A process for preparing an implant, comprising (a) admixing a composition comprising a bioresorbable polymer and a bioactive agent to form an admixture; and (b) processing the admixture into an implant at a temperature that is no greater than 70° C.
2. The process of aspect 1 wherein throughout admixing step (a), the bioresorbable polymer and the bioactive agent are dry.
3. The process of aspects 1 or 2 wherein step (a) is performed at or below 65° C.
4. The process of any preceding aspect, wherein forming the admixture in step (a) comprises spraying or coating a solution of the bioactive agent onto the bioresorbable polymer.
5. The process of any preceding aspect, wherein step (a) is performed at or below 60° C.
6. The process of any preceding aspect, wherein step (a) is performed at or below 50° C.
7. The process of any preceding aspect, wherein step (a) is performed at or below room temperature.
8. The process of any preceding aspect wherein step (a) is performed at or below 27° C.
9. The process of any preceding aspect wherein step (a) is performed at or below 25° C.
10. The process of any preceding aspect, wherein step (b) is performed at or below 60° C.
11. The process of any preceding aspect, wherein step (b) is performed at or below 50° C.
12. The process of any preceding aspect, wherein step (b) is performed at or below room temperature.
13. The process of any preceding aspect wherein step (b) is performed at or below 27° C.
14. The process of any preceding aspect wherein step (b) is performed at or below 25° C.
15. The process of any preceding aspect, wherein processing step (b) comprises extruding or molding the admixture.
16. The process of any preceding aspect, wherein the bioresorbable polymer is biodegradable.
17. The process of any preceding aspect, wherein the bioactive agent is solid.
18. The process of any preceding aspect, wherein the bioactive agent is powdered.
19. The process of any preceding aspect, wherein the bioactive agent is thermally labile.
20. The process of any preceding aspect, wherein the bioresorbable polymer is ground.
21. The process of any preceding aspect, wherein the bioresorbable polymer is present in the form of a microparticle.
22. The process of any preceding aspect wherein step (b) comprises melt pressing the admixture.
23. The process of any one of aspects 1-21 wherein step (b) comprises injection molding the admixture.
24. The process of any one of aspects 1-21 wherein step (b) comprises press extruding the admixture using a tablet press.
25. The process of any preceding aspect wherein prior to or during step (b), the bioresorbable polymer is contacted with a liquid, gaseous, or polymeric plasticizing agent.
26. The process of aspect 25 wherein the plasticizing agent comprises a low melt binder or a compression-based binder.
27. The process of aspect 25 wherein the plasticizing agent comprises an organic solvent.
28. The process of aspect 27 wherein the organic solvent comprises methylene chloride, ethylene chloride, chloroform, ethyl acetate, acetone, ethanol, methanol, isopropanol, butyl alcohol, or a mixture thereof.
29. The process of any one of aspects 1-24 wherein neither a plasticizing agent nor a solvent is used at any point in the process.
30. The process of any preceding aspect wherein the bioresorbable polymer comprises poly(lactide), poly(glycolide), poly(caprolactone), poly(lactide-co-glycolide), or a copolymer, combination, or blend thereof.
31. The process of any preceding aspect wherein the bioactive agent comprises an amino acid, a peptide, a protein, DNA, RNA, an aptamer, a receptor binding protein, or an antibody.
32. A process for preparing an implant, comprising (a) admixing, at or below room temperature, a bioresorbable polymer with a bioactive agent to produce an admixture; and (b) press extruding the admixture at or below room temperature.
33. The process of aspect 32 wherein during admixing step (a), the bioresorbable polymer and the bioactive agent are dry.
34. The process of aspects 32 or 33 wherein step (a) is performed at or below 27° C.
35. The process of any one of aspects 32-34 wherein step (a) is performed at or below 25° C.
36. The process of any one of aspects 32-35 wherein step (b) is performed at or below 27° C.
37. The process of any one of aspects 32-36 wherein step (b) is performed at or below 25° C.
38. The process of any one of aspects 32-37 wherein press extruding the admixture comprises tablet pressing the admixture.
39. The process of any one of aspects 32-38 wherein prior to or after step (a), the polymer is contacted with a liquid, gaseous, or polymeric plasticizing agent.
40. The process of aspect 39 wherein the plasticizing agent comprises a low melt binder or a compression-based binder.
41. The process of aspect 39 wherein the plasticizing agent comprises an organic solvent.
42. The process of aspect 41 wherein the organic solvent comprises methylene chloride, ethylene chloride, chloroform, ethyl acetate, acetone, ethanol, methanol, isopropanol, butyl alcohol, dimethyl sulfoxide, or a mixture thereof.
43. The process of any one of aspects 32-38 wherein neither a plasticizing agent nor a solvent is used at any point in the process.
44. The process of any one of aspects 32-43 wherein the bioactive agent is solid.
45. The process of any one of aspects 32-44 wherein the bioactive agent is powdered.
46. The process of any one of aspects 32-45 wherein the bioresorbable polymer is ground.
47. The process of any one of aspects 32-46 wherein the bioresorbable polymer is present in the form of a microparticle.
48. The process of any one of aspects 32-47 wherein the bioresorbable polymer is biodegradable.
49. The process of any one of aspects 32-48 wherein the bioresorbable polymer comprises poly(lactide), poly(glycolide), poly(caprolactone), poly(lactide-co-glycolide), or a copolymer, combination, or blend thereof.
50. The process of any one of aspects 32-49 wherein the bioactive agent comprises an amino acid, a peptide, a protein, DNA, RNA, an aptamer, a receptor binding protein, or an antibody.
51. A process for preparing an implant, comprising (a) cooling a bioresorbable polymer at or below −150° C.; (b) grinding the bioresorbable polymer; (c) admixing, at or below room temperature, the ground bioresorbable polymer with a solid powdered bioactive agent to produce an admixture; and (d) press extruding the admixture at or below room temperature to produce the implant.
52. The process of aspect 51 wherein during admixing step (c), the bioresorbable polymer and the solid powdered bioactive agent are dry.
53. The process of aspects 51 or 52 wherein step (c) is performed at or below 27° C.
54. The process of any one of aspects 51-53 wherein step (c) is performed at or below 25° C.
55. The process of any one of aspects 51-54 wherein step (d) is performed at or below 27° C.
56. The process of any one of aspects 51-55 wherein step (d) is performed at or below 25° C.
57. The process of any one of aspects 51-56 wherein the grinding is in a grinding mill that has been pre-cooled at or below −150° C. prior to step (b).
58. The process of any one of aspects 51-57 wherein prior to or during step (e), the bioresorbable polymer is contacted with a liquid, gaseous, or polymeric plasticizing agent.
59. The process of any one of aspects 51-58 wherein after step (b) and prior to or during step (d), the bioresorbable polymer is contacted with a liquid, gaseous, or polymeric plasticizing agent.
60. The process of aspects 58 or 59 wherein the plasticizing agent comprises a low melt binder or a compression-based binder.
61. The process of aspects 58 or 59 wherein the plasticizing agent comprises an organic solvent.
62. The process of aspect 61 wherein the organic solvent comprises methylene chloride, ethylene chloride, chloroform, ethyl acetate, acetone, ethanol, methanol, isopropanol, butyl alcohol, or a mixture thereof.
63. The process of any one of aspects 51-57 wherein neither a plasticizing agent nor a solvent is used at any point in the process.
64. The process of any one of aspects 51-63 wherein the bioresorbable polymer is present in the form of a microparticle.
65. The process of any one of aspects 51-64 wherein the bioresorbable polymer is biodegradable.
66. The process of any one of aspects 51-65 wherein the bioresorbable polymer comprises poly(lactide), poly(glycolide), poly(caprolactone), poly(lactide-co-glycolide), or a copolymer, combination, or blend thereof.
67. The process of any one of aspects 51-66 wherein the bioactive agent comprises an amino acid, a peptide, a protein, DNA, RNA, an aptamer, a receptor binding protein, or an antibody.
68. An implant prepared by the process of any preceding aspect.
69. An implant comprising (a) a bioresorbable polymeric matrix; (b) a bioactive agent dispersed in the matrix; and (c) from about 0.05 to about 5% of a plasticizing agent in the matrix.
70. The implant of aspect 69 wherein the plasticizing agent comprises a low melt binder or a compression-based binder.
71. The implant of aspect 69 wherein the plasticizing agent is polymeric.
72. The implant of aspect 69 wherein the plasticizing agent comprises an organic solvent.
73. The implant of aspect 72 wherein the organic solvent comprises methylene chloride, ethylene chloride, chloroform, ethyl acetate, acetone, ethanol, methanol, isopropanol, butyl alcohol, dimethyl sulfoxide, or a mixture thereof.
74. The implant of any one of aspects 69-73 wherein the bioresorbable polymer is present in the form of a microparticle.
75. The implant of any one of aspects 69-74 wherein the bioresorbable polymer is biodegradable.
76. The implant of any one of aspects 69-75 wherein the bioresorbable polymer comprises poly(lactide), poly(glycolide), poly(caprolactone), poly(lactide-co-glycolide), or a copolymer, combination, or blend thereof.
77. The implant of any one of aspects 69-76 wherein the bioactive agent comprises an amino acid, a peptide, a protein, DNA, RNA, an aptamer, a receptor binding protein, or an antibody.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Grinding (Milling) Procedure

Polymer particles were made of the desired size by grinding polymer with a Retsch Mill ZM 100 (Retsch, Dusseldorf, Germany). A 0.5-mm screen and 24-tooth rotor was used. The polymer was poly(lactide-co-glycolide) having 65 mol % lactide and 35 mol % glycolide and having ester end groups (hereinafter “65:35 PLG 4E”) (available from Birmingham Polymer Inc., Birmingham, Ala.). The polymer was chilled first in liquid nitrogen for 10 minutes before grinding. The Retsch Mill was also pre-chilled using liquid nitrogen. The frozen polymer was then continuously added to the Retsch Mill and ground using a mill speed of 18,000 rpm. After milling, the ground polymer was sieved through 90- and 300-micron sieves.

### Press Extrusion

Press extrusion of was performed using a Stoke's single station tablet press. The polymer was first ground and sieved to a defined particle size as described above. Bovine serum albumin, BSA, powder (available from SIGMA chemicals, MO) was mixed thoroughly with the polymer using a glass mortar and pestle. After mixing, approximately 15 mg was weighed onto a piece of weigh paper. The tablet press was set up with a 2.5 mm punch and die. The weighed drug/polymer blend was added to the die and the press was operated to produce a implant. The implant was carefully removed from the die by allowing the bottom punch to push the compressed implant out of the die. The implant was removed from the punch using tweezers.

The pressure of the Tablet Press punches was adjusted until pressed implants prepared with the un-treated polymer (no solvent or plasticizing agent used at any point in the process) produced implants having a minimum hardness (breaking pressure) of 25 lbs as determined by the method described below. The implant crush pressure for Examples 1, 2, and 5 are listed in Tables 1 and 2. The Tablet Press pressure used to prepare the Examples 1, 2, and 5 was then used to prepare the Test implants (implants prepared from the BSA-polymer blends that were contacted with the solvent). Measurement of implant hardness of the test implants was not performed because the residual solvent in these implants caused them to be plasticized (softened) so no comparable determination could be made in these samples as compared to Examples 1, 2, and 5.

### Implant Hardness

The implant crush pressure was measured using a PFIZER hardness tester. The implant is placed in the jaws of the tester and the tester was operated until the implant broke or crushed. The pressure at which the implant breaks or crushed is recorded at the tablet hardness.

### Solvent Treatment of Samples

Examples 3 and 4 were prepared by treating implants from Examples 1 and 2, respectively, to a solvent-treatment operation. In a this operation, an implant was held with tweezers and dipped in a bath of methylene chloride for 3 seconds and then removed. The dipped implant was allowed to dry on a piece of Teflon for one hour.

BSA Implant with Plasticized Polymer

After mixing the BSA and the polymer with a mortar and pestle, the mixture was added to the die. Either 20 or 50 uL of methylene chloride was then added to the die to plasticize the polymer. After 30 seconds, the implant was prepared using the tablet press as described previously. Examples 6-8 were prepared using 50 uL of added methylene chloride while examples 9-11 were prepared using 20 uL of added methylene chloride. After removal from the tablet press, the pressed implants were allowed to dry on a sheet of Teflon film for one hour.

BSA Content Analysis of Pressed implants

A Pierce BCA protein assay kit (from Pierce Biotechnology Inc.; Rockford Ill.) was used to analyze the BSA content of the pressed implants. Triplicate samples were prepared and analyzed as follows. An individual implant was accurately weighed into a test tube to which was added 2 mL of 1 N NaOH. The test tube contents were allowed to dissolve over approximately 18 hours. After which time, 2 mL of phosphate-buffered saline (PBS), pH 7.4, was added, and the pH was adjusted to pH 7 using phosphoric acid. The contents were then analytically transferred to a 10 mL volumetric flask, which was then diluted to volume with PBS (phosphate-buffered saline).

Protein analysis was then carried out according to the instructions of the Pierce BCA protein assay kit. Standards were prepared from the BSA powder used to prepare the implants. Extraction efficiency was verified using spiked-control samples containing similar ratios of drug and polymer which were treated to the same extraction steps that were used for analysis of the BSA implants. Loading values for individual examples are shown in Tables 1 to 3.

In Vitro Release of BSA from Pressed Implants

Individual implants were separately weighed into a 20-mL scintillation vial followed by adding 10 mL of (PBS) receiving fluid. Triplicate samples were prepared. Next the vials were placed in a 37° C. shaker bath shaking at a speed of 50 rpm. At the appropriate time point, 2 mL of buffer was removed from a vial. Then 2 mL of fresh receiving fluid was placed into the vial before returning it to the shaker bath. The removed sample was quantified for BSA using the BCA protein Kit mentioned above. In vitro release kinetics, cumulative percent of BSA released was reported. The resulting in vitro release data are shown in Tables 1 to 3.

BSA Release from Samples

Cumulative in vitro release of BSA from un-treated samples (Examples 1, 2, and 5 in Tables 1 and 2) show that the compressed implants made from the un-treated polymer release quickly (Examples 1, 2, and 5) with 90% or more release achieved within about 1 to 3 days. A post processing solvent-treatment step was useful in slowing down the relative release patterns (Examples 3 and 4 as compared to Examples 1 and 2); after such treatment, cumulative release reached 90% (or more) within 3 to 4 days.

By comparison, compressed implants prepared using the plasticized polymer showed release that extended out to (and beyond) the final 7-day time point used in this study (Examples 6-8 were prepared using 50 microliter dichloromethane, Examples 9-11 were prepared using 20 microliter dichloromethane).

TABLE 1
Implants made with un-treated polymer-drug blends

| | | Implant | Tablet | 1 Day | 2 Day | 3 Day | 4 Day | 5 Day | 7 Day |
|---|---|---|---|---|---|---|---|---|---|
| | BSA | Forming | breaking | In Vitro | In Vitro | In Vitro | In Vitro | In Vitro | In Vitro |
| Example | loading | Conditions | pressure | Release | Release | Release | Release | Release | Release |
| 1 | 20% | Pressed using | 28 lbs | 90% | ~100% | | | | |
| | | un-treated | | | | | | | |
| | | polymer (no | | | | | | | |
| | | solvent) | | | | | | | |
| 2 | 30% | Pressed using | 26 lbs | 60% | 82% | ~100% | | | |
| | | un-treated | | | | | | | |
| | | polymer (no | | | | | | | |
| | | solvent) | | | | | | | |
| 3 | 20% | Example 1 | | 52% | 75% | 92% | | | |
| | | implant that | | | | | | | |
| | | was solvent | | | | | | | |
| | | dip-coated | | | | | | | |
| 4 | 30% | Example 2 | | 37% | 54% | 75% | 93% | | |
| | | implant that | | | | | | | |
| | | was solvent | | | | | | | |
| | | dip-coated | | | | | | | |

TABLE 2
Test Implants made with plasticized polymer-drug blend (50 microliter dichloromethane)

| | | | Tablet | | | | | | |
|---|---|---|---|---|---|---|---|---|---|
| | | | breaking | 1 Day | 2 Day | 3 Day | 4 Day | 5 Day | 7 Day |
| Example | loading | Conditions | pressure | Release | Release | Release | Release | Release | Release |
| 5 | 5% | Pressed using un- | 30 lbs | 92% | | | 94% | | |
| | | treated polymer | | | | | | | |
| | | (no solvent) | | | | | | | |
| 6 | 10% | Plasticized with | | 35% | | | 52% | 66% | 74% |
| | | dichloromethane | | | | | | | |
| 7 | 5% | Plasticized with | | 27% | 35% | 42% | | | 62% |
| | | dichloromethane | | | | | | | |
| 8 | 1% | Plasticized with | | 15% | 25% | 37% | | | 50% |
| | | dichloromethane | | | | | | | |

TABLE 3
Test Implants made with plasticized polymer-drug blend (20 microliter dichloromethane)

| | | | 1 Day | 2 Day | 3 Day | 4 Day | 6 Day | 7 Day |
|---|---|---|---|---|---|---|---|---|
| Example | loading | Conditions | Release | Release | Release | Release | Release | Release |
| 9 | 10% | Plasticized with | 28% | 36% | 68% | 77% | 85% | 86% |
| | | dichloromethane | | | | | | |
| 10 | 5% | Plasticized with | 37% | 45% | 46% | 55% | 57% | 64% |
| | | dichloromethane | | | | | | |
| 11 | 1% | Plasticized with | 21% | 27% | 42% | 62% | 68% | 75% |
| | | dichloromethane | | | | | | |

Various modifications and variations can be made to the devices, compositions, and methods described herein. Other aspects of the devices, compositions, and methods described herein will be apparent from consideration of the specification and practice of the devices, compositions, and methods disclosed herein. It is intended that the specification and examples be considered as exemplary.

What is claimed is:

1. A process for preparing an implant, comprising (a) admixing a composition comprising a bioresorbable polymer and a bioactive agent to form an admixture; and (b) processing the admixture into an implant at a temperature that is no greater than 70° C.

2. The process of claim 1 wherein step (a) is performed at or below 65° C.

3. The process of claim 1 wherein forming the admixture in step (a) comprises spraying or coating a solution of the bioactive agent onto the bioresorbable polymer.

4. The process of claim 1 wherein step (a) is performed at or below 25° C.

5. The process of claim 1 wherein step (b) is performed at or below 25° C.

6. The process of claim 1 wherein step (b) comprises press extruding the admixture using a tablet press.

7. The process of claim 1 wherein prior to or during step (b), the bioresorbable polymer is contacted with a liquid, gaseous, or polymeric plasticizing agent.

8. The process of claim 1 wherein the bioresorbable polymer comprises poly(lactide), poly(glycolide), poly(caprolactone), poly(lactide-co-glycolide), or a copolymer, combination, or blend thereof.

9. A process for preparing an implant, comprising (a) admixing, at or below room temperature, a bioresorbable polymer with a bioactive agent to produce an admixture; and (b) press extruding the admixture at or below room temperature.

10. The process of claim 9 wherein prior to or after step (a), the polymer is contacted with a liquid, gaseous, or polymeric plasticizing agent.

11. The process of claim 10 wherein the plasticizing agent comprises an organic solvent.

12. A process for preparing an implant, comprising (a) cooling a bioresorbable polymer at or below −150° C.; (b) grinding the bioresorbable polymer; (c) admixing, at or below room temperature, the ground bioresorbable polymer with a solid powdered bioactive agent to produce an admixture; and (d) press extruding the admixture at or below room temperature to produce the implant.

13. The process of claim 12 wherein step (c) is performed at or below 25° C.

14. The process of claim 12 wherein prior to or during step (e), the bioresorbable polymer is contacted with a liquid, gaseous, or polymeric plasticizing agent.

15. The process of claim 12 wherein after step (b) and prior to or during step (d), the bioresorbable polymer is contacted with a liquid, gaseous, or polymeric plasticizing agent.

16. The process of claim 14 wherein the plasticizing agent comprises a low melt binder or a compression-based binder.

17. The process of claim 14 wherein the plasticizing agent comprises an organic solvent.

18. The process of claim 17 wherein the organic solvent comprises methylene chloride, ethylene chloride, chloroform, ethyl acetate, acetone, ethanol, methanol, isopropanol, butyl alcohol, or a mixture thereof.

19. An implant comprising (a) a bioresorbable polymeric matrix; (b) a bioactive agent dispersed in the matrix; and (c) from about 0.05 to about 5% of a plasticizing agent in the matrix.

20. The implant of claim 19 wherein the bioresorbable polymer is present in the form of a microparticle.
